(19) United States
(12) Patent Application Publication
(10) Pub. No.: US 2022/0162151 A1
(43) Pub. Date: May 26, 2022

(54) PROCESS AND DEVICE FOR PRODUCING NITROBENZENE

(71) Applicants: Covestro Deutschland AG, Leverkusen (DE); Covestro Intellectual Property GmbH & Co. KG, Leverkusen (DE)

(72) Inventors: Thomas Knauf, Dormagen (DE); Murat Kalem, Neuss (DE); Christian Drumm, Frohnhofen (DE); Alexandre Racoes, Krefeld (DE)

(21) Appl. No.: 17/602,333

(22) PCT Filed: Apr. 14, 2020
(86) PCT No.: PCT/EP2020/060437

§ 371 Date: Oct. 8, 2021

(30) Foreign Application Priority Data
Apr. 17, 2019 (EP) 19169872.9
Mar. 23, 2020 (EP) 20164916.7

(51) Int. Cl. C07C 201/08; C07C 201/16

(52) U.S. Cl. CPC C07C 201/08; C07C 201/16

(57) ABSTRACT

The invention relates to a continuously operating process for producing nitrobenzene, comprising the following steps: a) nitriding benzene in adiabatic conditions with sulfuric acid and nitric acid, using a stoichiometric excess of benzene in relation to the nitric acid, in multiple parallel reactors; b) first combining the raw process products of the nitridation from the parallel reactors to form a mixed flow in a device provided specifically for this purpose, then separating the mixed flow into a sulfuric acid phase and a nitrobenzene phase in a downstream phase separation apparatus; and c) processing the nitrobenzene phase, obtaining nitrobenzene. The invention also relates to a production plant suitable for carrying out the claimed process.

The invention relates to a continuously operated process for the preparation of nitrobenzene, comprising the steps of: a) nitrating benzene under adiabatic conditions with sulfuric acid and nitric acid using a, based on nitric acid, stoichiometric excess of benzene in a plurality of reactors operated in parallel; b) first combining the crude process products of the nitration from the reactors operated in parallel to give a mixed stream in an apparatus provided specifically for this purpose, followed by separating the mixed stream into a sulfuric acid phase and a nitrobenzene phase in a downstream phase separation apparatus; c) working up the nitrobenzene phase to obtain nitrobenzene.

The invention also relates to a production plant suitable for performing the process according to the invention.

The nitration of benzene with nitric acid in the presence of sulfuric acid to give nitrobenzene and water has already been the subject of numerous publications and patent applications. A distinction is made here between two basic types of processes, the “isothermal” mode and the “adiabatic” mode.

In the isothermal mode, the (considerable) heat of reaction from the nitration is removed as far as possible by indirect cooling using a heat transfer medium.

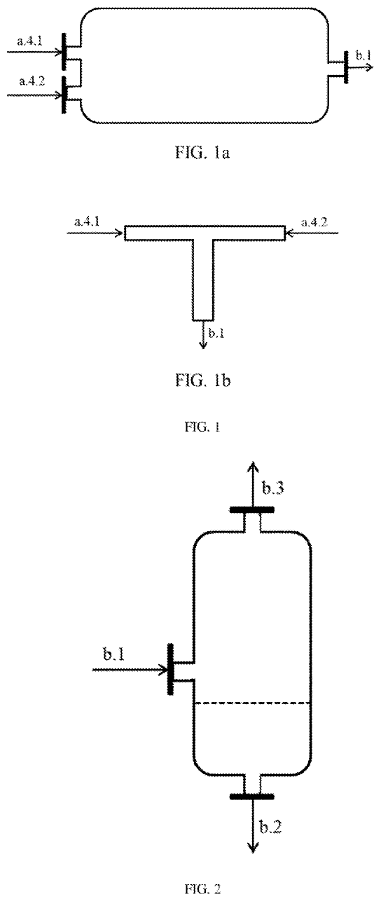

An isothermal process for preparing nitrobenzene, in which a reaction loop is used, is described in U.S. Pat. No. 3,092,671 B1. In this process—see FIG. 1 and the explanatory passages of text—benzene and a mixture of sulfuric and nitric acid are pumped through a nitration reactor (4) by means of a centrifugal pump (1) and reacted. The nitration reactor (4) is designed as a heat exchanger in which the reaction temperature is maintained between 120° F. (48.9° C.) and 150° C. (65.6° C.) by thermostatting (cf. the examples and patent claim 4). The reaction product obtained (comprising not only nitrobenzene but also an acid phase) is partly recycled—without removal of the acid phase—into the reaction. The remaining part of the liquid product mixture is passed into a phase separation apparatus (not shown in FIG. 1), in which phase separation into crude nitrobenzene and acid phase takes place.

In the adiabatic mode—which is currently more common and is also used in the present invention—cooling of the nitration reactor is omitted and therefore the exothermicity of the reaction, once unavoidable heat losses are disregarded, is reflected quantitatively in the temperature difference between the temperature on entry into the nitration reactor and the temperature of the completely converted product mixture (what is known as an adiabatic jump in temperature). In order that this temperature rise does not become excessive, adiabatically operated processes typically employ a very large sulfuric acid excess. A continuous process for preparing nitrobenzene by means of an adiabatically operated nitration of benzene using a mixture of sulfuric and nitric acid (so-called mixed acid) was first claimed in 1941 in U.S. Pat. No. 2,256,999, which described an arrangement of four stirred tanks which are arranged in parallel and are successively supplied with the reactants. A circulation regime as described in U.S. Pat. No. 3,092,671 B1 is not possible in such an adiabatic mode without relinquishing the economic advantages of this process at least to a certain extent, since here as a consequence of the adiabatic temperature jump the reaction product has a temperature which is much higher than the temperature of the mixed reactants before the start of the reaction. The comparatively high temperature of the reaction product is (after separation thereof into an acid phase and a nitrobenzene phase) needed for the flash evaporation of water contained in the acid phase. Recycling part of the reaction product prior to phase separation, as described in U.S. Pat. No. 3,092,671 B1, would necessitate cooling of the recycled fraction, which would impair the energy balance of the process and hence the economic viability thereof.

The reaction in adiabatic operating mode is generally conducted in such a way that the nitric acid and sulfuric acid are combined to give what is called the nitrating acid (also called mixed acid). Benzene is metered into this nitrating acid. This procedure is also preferably used in the process according to the invention. The reaction products are essentially water and nitrobenzene. In the nitration reaction, benzene, based on the molar amount of nitric acid, is used at least in a stoichiometric amount, but preferably in a 2% to 10% excess, so that the process product obtained in the nitration is essentially free from nitric acid. This process product is fed to a phase separation apparatus in which two liquid phases form, an organic phase and an aqueous phase. The organic phase is referred to as crude nitrobenzene and essentially consists of nitrobenzene, benzene and a certain amount of water and sulfuric acid dissolved in the nitrobenzene. The aqueous phase is referred to as waste acid and consists essentially of water, sulfuric acid and nitrobenzene dissolved in the sulfuric acid. In addition to these liquid constituents, the process product of the nitration also contains gaseous components, specifically firstly organic components such as evaporated benzene and low-boiling, nonaromatic secondary components (usually referred to as low boilers), and secondly inorganic components such as in particular nitrous gases (NOₓ), formed as a result of side reactions of the nitric acid used. According to the prior art, these gaseous components separate from the two liquid phases in the phase separation apparatus and are discharged via a separate outlet as offgas stream. This offgas stream from the phase separation apparatus can be combined with the various offgas streams from other parts of the plant and worked up, where, as described in patent application EP 2 719 682 A1, benzene can be recovered and the nitrous gases can be converted to nitrous acid. In this way, the recovered benzene and the nitrous acid can be recycled and resupplied to the nitration.

The crude nitrobenzene formed in the reaction apparatuses and separated off from the acid phase in the phase separation apparatus is subjected to washing and a distillative workup according to the prior art. A characteristic feature of this workup is that unconverted excess benzene, after the wash, is separated off from nitrobenzene in a final distillation as “return benzene”. This return benzene, which—in addition to the gas phase discharged in the phase separation apparatus—also contains a portion of the low-boiling, nonaromatic organic compounds (low boilers), is reused in the nitration reaction.

The international patent application WO 2015/197521 A1 relates to a process for continuously preparing nitrobenzene by nitrating benzene with a mixture of nitric acid and sulfuric acid, in which, during a production shutdown, rather than shutting the whole production plant down, the production plant is run wholly or at least partly “in circulation”. This patent application further relates to a plant for preparation of nitrobenzene and to a method of operating a plant for preparation of nitrobenzene. The plant for preparation of nitrobenzene can include two or more nitration reactors connected in parallel or in series.

Patent application US 2017/152210 A1 (also published as WO 2015/197522 A1) relates to processes for preparing chemical products in which the feedstock(s) is/are converted to give a chemical product or a chemical composition, and also to plants for performing such processes. The processes and the plants have the feature that, during a production interruption, at least one feedstock is not introduced into the reaction and operation of the plant parts not affected by inspection, maintenance, repair or cleaning measures is continued in what is known as circulation mode. The plants described can have parallel- or series-connected reactors. One example mentioned of processes and plants to which the described invention can be applied is nitrobenzene preparation.

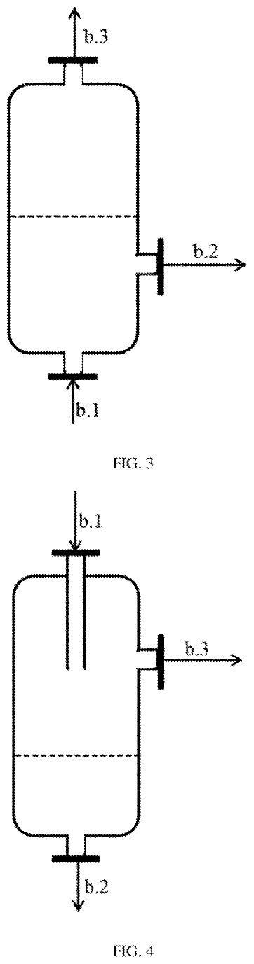

Patent application EP 0 696 574 A1 is concerned with a process for hydrogenation of nitroaromatics to aromatic amines in the gas phase over fixed catalysts, wherein heat is neither externally supplied to nor extracted from the catalyst, that is to say that the process is operated adiabatically. FIG. 2 shows a production plant with three parallel-connected reactors (II, III and IV). The reaction products (6, 7, 8) of the three reactors are combined in a common line and after cooling in a heat exchanger (V) are passed into a distillation column (VIII) for vapor generation. A water/aniline vapor mixture (12) is obtained at the top of the distillation column (VIII) and is condensed in a condenser (IX). A first portion of the condensate (13) is returned as reflux back to the distillation column (VIII), while a second portion is passed to a separation vessel (X). In this separation vessel, water-containing aniline (14) is separated off from aniline-containing water (16). The water-containing aniline (14) is combined with the bottom stream (11) of the distillation column (VIII), which also contains water-containing aniline, and sent to further workup. The fixed catalysts are attached in the form of catalyst beds on or between gas-permeable walls. The use of honeycombs or corrugated layers which have been rendered catalytically active by application of suitable metal compounds, instead of catalyst beds, is likewise possible. Such reactors configured for gas-phase reactions with fixed catalysts are not suitable for the nitration of benzene with nitric acid in the presence of sulfuric acid, where the sulfuric acid—just like the other reactants as well—flows through the reactor and, besides its action as catalyst, also serves to absorb the heat of reaction.

The Chinese patent application CN 1789235 A is concerned with the use of a tubular reactor in nitration reactions.

Patent application DE 10 2009 005324 A1 is concerned with the problems which can accompany the high content of low boilers in the return benzene and describes, in this context, a process for preparing nitrobenzene by adiabatic nitration of benzene, in which the benzene/low boiler mixture obtained during the purification of the nitrobenzene is recycled to the nitration and the crude nitrobenzene is separated off from the sulfuric acid after the reaction under pressure.

The treatment of the offgas from the adiabatically performed nitration reaction with respect to nitrous gases is described in EP 0 976 718 A2. The offgas from the acid circuit and from the crude nitrobenzene is taken off, combined and sent via an NOₓ absorber in order to recover dilute nitric acid, which is returned into the reaction. The circulated sulfuric acid is concentrated in a flash evaporator and very substantially freed of organics. Traces of high-boiling organics such as nitrobenzene, dinitrobenzene and nitrophenols remain in the circulated acid and hence are also returned to the reaction.

Patent application WO 2014/016292 A1 describes how the nitrobenzene process may be better started up, by keeping the content of aliphatic organic compounds in the feed benzene during the startup time low (proportion by mass of less than 1.5%). This is achieved by adjusting the ratio of fresh benzene to return benzene during the startup time depending in particular on the purity of the return benzene, such that the stipulated maximum content of aliphatic organic compounds in the feed benzene is not exceeded. The proportion of return benzene during the startup time can also be zero; in this case only fresh benzene of sufficient purity is supplied to the nitration reactor during the startup time. Patent application WO 2014/016289 A1 describes how the continuous nitration of benzene to nitrobenzene in regular operation can be improved by limiting the content of aliphatic organic compounds in the feed benzene to a proportion by mass of less than 1.5%. In one embodiment, this is achieved by discharging low boilers with the gas phase of the phase separation apparatus. Both patent applications relate in particular to an improved product quality and optimized washing of the crude nitrobenzene; the influence of low boilers in the phase separation apparatus is not dealt with, however.

The phase separation apparatus (also called decanter) does not only have the important task of separating the process product of the nitration into an aqueous acidic phase and an organic phase containing crude nitrobenzene. In addition and as already mentioned, a gas phase containing benzene, low boilers and nitrous gases is also drawn off in the phase separation apparatus. A sufficiently high residence time therefore needs to be provided in the phase separation apparatus so that these physical processes (separation of the crude process product of the nitration into two liquid phases and a gas phase) can be performed without negatively impacting the production capacity of the plant. Due to the presence of the gas phase in the apparatus, the separation apparatus has to be designed much larger than would be the case for a pure liquid-liquid separation.

The efficiency of gas-liquid or liquid-liquid phase separation apparatuses can be increased according to the prior art by means of particular internals or a particular configuration of the entrance into the apparatus. This also applies to the phase separations in the nitrobenzene process (phase separation after the reaction and phase separations in the context of the washes). Internals such as plate internals, knitted meshes, lamellae and random packings may homogenize and stabilize the flow and enlarge the surface area, so that phenomena such as coalescence and the separation of droplets and bubbles proceed more quickly. Entry into the phase separation apparatus can be via baffles or deflecting plates which stabilize the flow or direct it towards the apparatus wall with the aim of increasing the residence time in the apparatus and hence of improving the separating efficiency. Established variants are described for example in Gulf Equipment Guides, Gas-Liquid and Liquid-Liquid Separators, chapter 3.5 (Vessel Internals) on pages 84 to 89, year 2009, by Maurice Stewart and Ken Arnold, and in Fundamentals of Natural Gas Processing, chapter 5, pages 105 to 117, year 2011, by Arthur J Kidnay, William R Parrish and Daniel G. McCartney. The variants described in the cited literature are explained in part using the example of gas-liquid phase separations, but are, as concerns the fundamental principles, also usable for liquid-liquid or triphasic gas-liquid-liquid separations. The disadvantage with the prior art processes is that deposits and fouling may occur as a result of the flow stabilization and the nature of the internals. For example, knitted meshes and lamellae become clogged over time and deposits form on the plates. The internals can be damaged by pressure shocks or excessively high flow velocities. Due to the corrosive media, the phase separation apparatuses are usually manufactured from enamel on the inside. The apparatuses can be damaged by the internals and maintenance or servicing of the apparatuses becomes more expensive.

Operational practice has shown that problems can arise time and again in the phase separation of the crude process product of the nitration. These manifest, for example, in inadequate phase separation (e.g. entrainment of organics into the acid phase or formation of black deposits). These problems then arise to a greater degree when the crude process products of two or more, in particular independently controllable, nitration reactors operated in parallel, that is to say when carrying out the reaction in two or more reaction lines (also referred to as reaction trains) operated in parallel, are passed into a common phase separation apparatus. This approach is not uncommon in practice. A multi-line reaction in conjunction with a single-line workup has often proven to be the best compromise between the requirements of minimizing investment costs on the one hand and maximizing flexibility in production on the other.

There was therefore a need for further improvement in the preparation of nitrobenzene in multiple reaction lines operated in parallel, in particular as concerns the efficiency of separation of the reaction product of the nitration into two liquid phases and a gaseous phase. It would be desirable in particular to configure as optimally as possible the discharge of the gaseous fraction and the separation of the two liquid phases from each other, both as concerns the quality of the separation and the process-engineering and apparatus configuration. Different loads and reaction conditions on the individual reaction lines should also not impair the separating efficiency. This need is accommodated by the present invention both from a process engineering viewpoint and in terms of apparatus.

It has surprisingly been found that problems observed time and time again in the liquid-liquid phase separation are associated with the combining of the process products of the individual reaction lines in a common phase separation apparatus (that is to say the performance of the mixing and phase separation in one and the same apparatus), and that these problems can be solved or at least appreciably reduced when the mixing is effected separately from the phase separation. As demand for the product nitrobenzene fluctuates, for example, the throughputs in the individual reaction lines are varied, meaning that different amounts pass from the individual lines into the phase separation apparatus, which can lead to reduced separation efficiency. Since the individual lines usually flow into the separation apparatus at different locations, the different amounts result in different local velocities and undesirable crossflows and backflows which negatively impact the separating efficiency, and in the event of an excessively high load on a reaction line excessive turbulence and further losses of efficiency also result. Different reaction conditions such as pressures, temperatures and concentrations also lead to mixing and equalization processes in the apparatus, which have to proceed in parallel and slow the demixing of the liquid phases. In addition, problems can arise as a result of the simultaneous discharge of the gas phase. Depending on the proportion of the gaseous phase, the presence thereof can lead to much greater velocities and turbulence in the liquid phases in the phase separation apparatus, which impedes the separation of the two liquid phases. In the event of fluctuating proportions or an increase in the gas phase, there may therefore be inadequate separation of the liquid phases, meaning that even greater proportions of organics may pass into the aqueous acidic phase. The following conclusions have been drawn: The presence of the gas phase in the separator generally leads to high velocities (and also to higher velocities of the liquid phases), since the gas phase moves at far greater velocities on account of the lower density compared to the liquid. Furthermore, the presence of the gas phase and the resulting triphasic gas-liquid-liquid separation also impairs the separating efficiency of the two liquid phases. Rising gas bubbles impede demixing of the liquid phases since mixing is constantly occurring again at the liquid-liquid phase boundary and the liquid phase with the higher density can be entrained together with the gas bubbles into the liquid phase with the lower density.

The present invention therefore firstly provides a process for the continuous preparation of nitrobenzene, comprising the steps of
a) nitrating benzene under adiabatic conditions with sulfuric acid and nitric acid using a, based on nitric acid, stoichiometric excess of benzene in n parallel-connected reactors, where n is a natural number in the range from 2 to 5, so that n process products containing nitrobenzene, benzene and sulfuric acid (henceforth also referred to as crude process products of the nitration) are obtained;
   (i) combining the n process products containing nitrobenzene, benzene and sulfuric acid into one mixed stream containing nitrobenzene, benzene and sulfuric acid, optionally additionally comprising a depletion of gaseous constituents (α) after, (β) before or (γ) during the combining operation,
   (ii) introducing the mixed stream, which may have been depleted of gaseous constituents, (either in its unmodified entirety or divided into two or more, in particular n, preferably 2 to 3, substreams) into a phase separation apparatus in which the mixed stream is separated into a liquid aqueous sulfuric acid phase and a liquid organic nitrobenzene phase;
c) working up the nitrobenzene phase from step b) to obtain nitrobenzene;
   and optionally
d) evaporating water from the sulfuric acid phase obtained in step b) to obtain a concentrated sulfuric acid phase, and using concentrated sulfuric acid phase as a constituent of the sulfuric acid used in step a).

The present invention secondly provides a production plant for performing the process according to the invention for the continuous preparation of nitrobenzene, wherein the production plant comprises the following apparatuses:
a) n parallel-connected reactors for the adiabatic nitration of benzene with sulfuric acid and nitric acid using a, based on nitric acid, stoichiometric excess of benzene, where n is a natural number in the range from 2 to 5, to obtain n process products containing nitrobenzene, benzene and sulfuric acid;
b) (i) arranged downstream of the reactors of a), an apparatus for combining the n process products containing nitrobenzene, benzene and sulfuric acid into one mixed stream containing nitrobenzene, benzene and sulfuric acid,
   (ii) arranged downstream of the apparatus for combining the n process products containing nitrobenzene, benzene and sulfuric acid, a phase separation apparatus for separating the mixed stream obtained into a liquid aqueous sulfuric acid phase and a liquid organic nitrobenzene phase;
c) an apparatus for working up the liquid organic nitrobenzene phase from b)(ii) to give nitrobenzene, this apparatus in particular comprising the following devices:
   (i) devices for washing the liquid organic nitrobenzene phase and devices for removing unconverted benzene;
   (ii) devices for recycling removed benzene from c)(i) into the reactor of a) as a constituent of the benzene used there;
d) optionally, devices for concentrating the sulfuric acid phase from b)(ii) by evaporating water and devices for recycling concentrated sulfuric acid phase thus obtained into the n reactors from a).

In the terminology of the present invention, the term “gaseous secondary components” encompasses at least the low boilers already mentioned hereinabove, low boilers being understood as being all nonaromatic, organic secondary components of the process product of the nitration (=step a)) which have boiling points at standard pressure (1013 mbar) lying below that of nitrobenzene. Typical low boilers are n-heptane, dimethylcyclopentane, 3-ethylpentane, cyclohexane, the isomeric dimethylpentanes, n-hexane, cyclopentane, n-pentane, trimethylcyclopentane, methylcyclohexane, ethylcyclopentane and octane. In addition, inorganic secondary components may also be present, in particular such as the nitrous gases already mentioned.

In the process according to the invention, the mixed stream obtained in step b)(i) is fed to the phase separation of step b)(ii), specifically without recycling part of this mixed stream into the reaction of step a). The same applies for the n process products containing nitrobenzene, benzene and sulfuric acid prior to the combining thereof to form a mixed stream; these are not recycled into step a), either. A reaction loop, as described in the prior art for isothermal processes, is not subject matter of the process according to the invention. The same applies, of course, to the production plant according to the invention; this does not have devices for recycling the n process products containing nitrobenzene, benzene and sulfuric acid or the mixed stream obtained in the apparatus for combining the n process products containing nitrobenzene, benzene and sulfuric acid [b)(i)] into one, a plurality of, or all of the n reactors [a)].

In the appended drawings:

FIG. 1 shows two possible configurations (FIG. 1a and FIG. 1b) of the apparatus for combining the n process products containing nitrobenzene, benzene and sulfuric acid;

FIG. 2 shows a vertically arranged gas separator with lateral feed of the input stream (b.1) and discharge of the gas phase (b.3) at the top and discharge of the liquid phase (b.2) at the bottom;

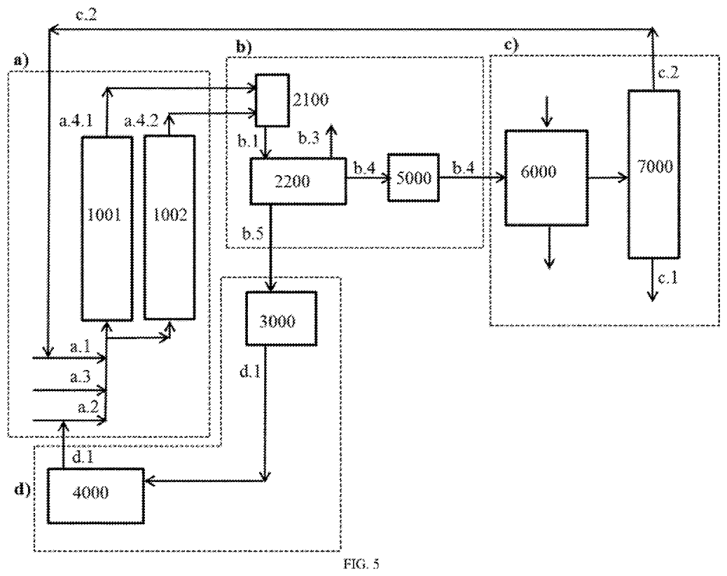

FIG. 3 shows a vertically arranged gas separator with feed of the input stream (b.1) at the bottom and discharge of the gas phase (b.3) at the top and discharge of the liquid phase (b.2) from the side;

FIG. 4 shows a vertically arranged gas separator with feed of the input stream (b.1) at the top and discharge of the gas phase (b.3) from the side and discharge of the liquid phase (b.2) at the bottom;

FIG. 5 shows a possible configuration of a production plant according to the invention for the case where n=2;

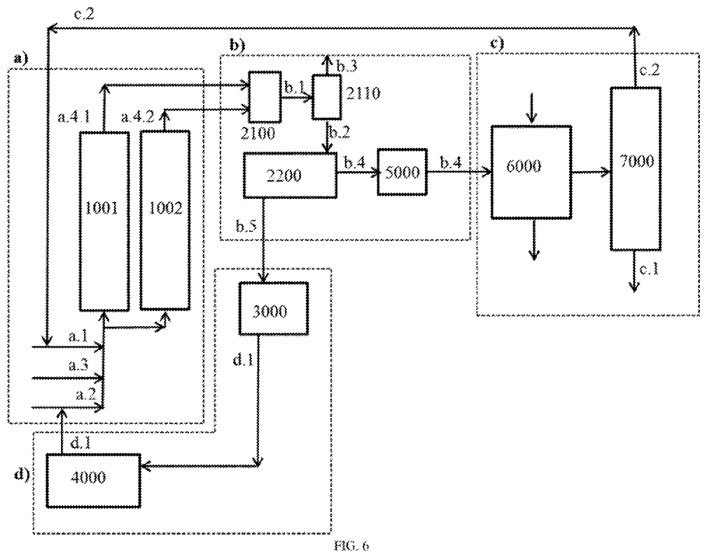

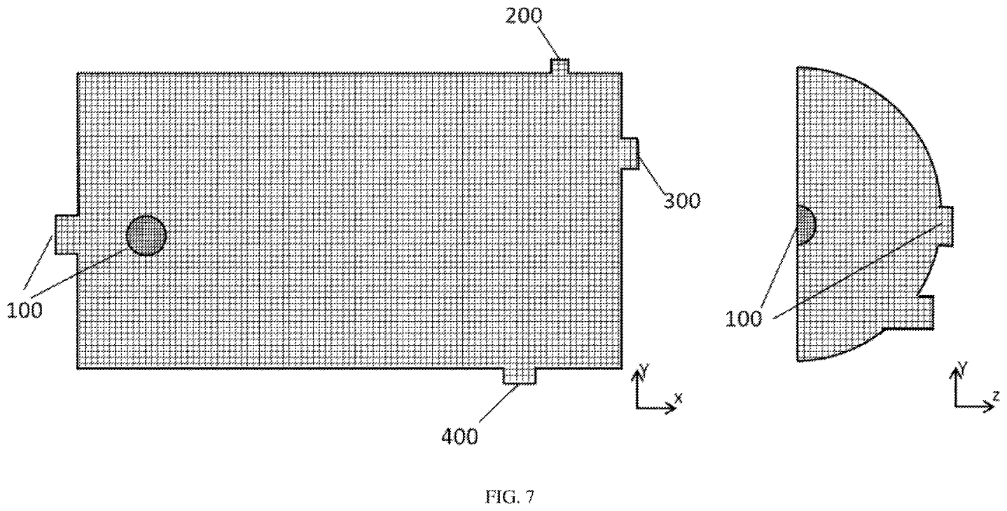

FIG. 6 shows a possible configuration of a production plant according to the invention for the case where n=2 in conjunction with an additional gas-liquid separation;

FIG. 7 shows the grid used for the Computational Fluid Dynamics (CFD) calculations of the examples;

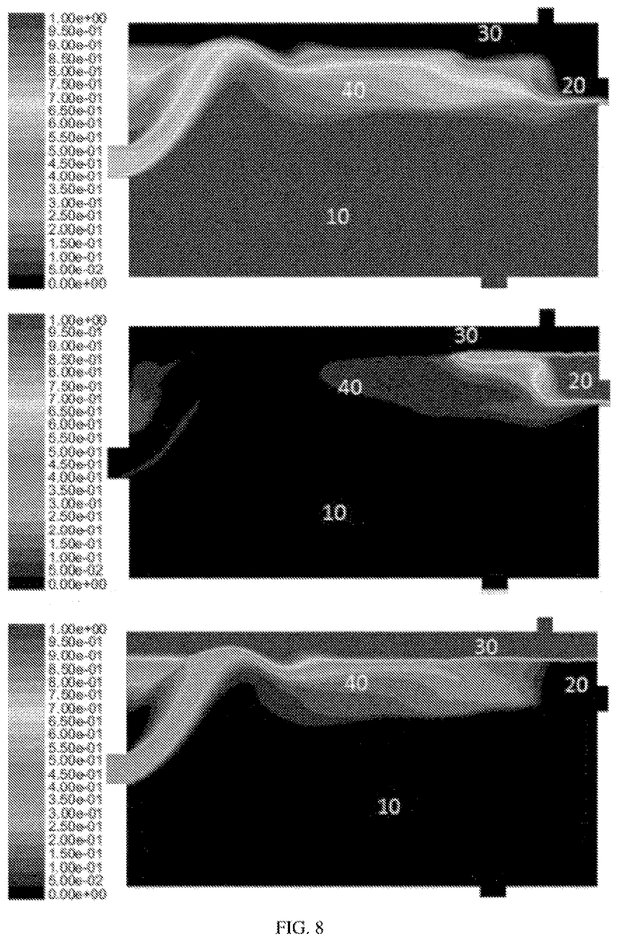

FIG. 8 shows the volume fractions of the three phases (top: aqueous phase, middle: organic phase, bottom: gas phase) in the CFD simulation of example 1 (comparative example; without combining (=homogenization) of the crude process products and without degassing before entry into the phase separation apparatus);

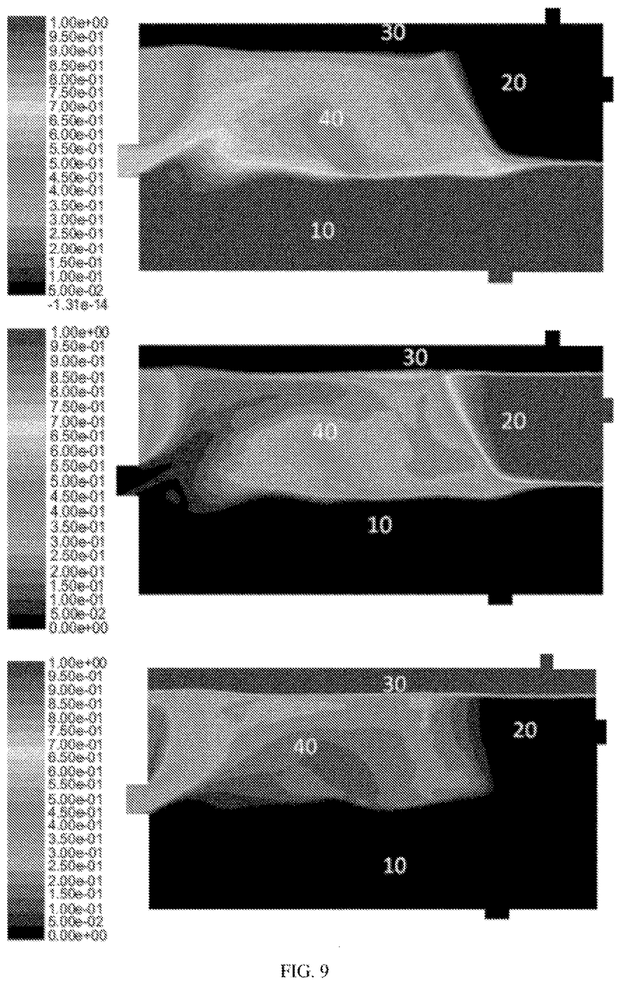

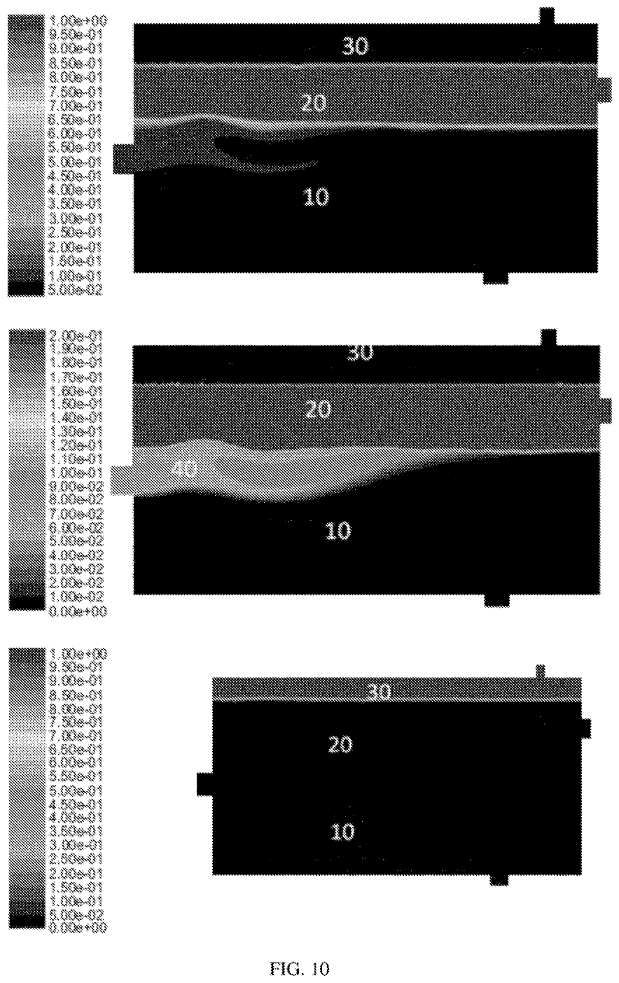

FIG. 9 shows the volume fractions of the three phases (top: aqueous phase, middle: organic phase, bottom: gas phase) in the CFD simulation of example 2 (example according to the invention; with combining (=homogenization) of the crude process products and without degassing before entry into the phase separation apparatus);

FIG. 10 shows the volume fractions of the three phases (top: aqueous phase, middle: organic phase, bottom: gas phase) in the CFD simulation of example 3 (example according to the invention; with combining (=homogenization) of the crude process products and with degassing before entry into the phase separation apparatus).

There follows firstly a brief summary of various possible embodiments.

In a first embodiment of the process according to the invention, which can be combined with all other embodiments, the workup of the nitrobenzene phase in step c) comprises the following:
(i) washing the nitrobenzene phase and removing unconverted benzene,
(ii) using removed benzene as a constituent of the benzene used in step a).

In a second embodiment of the process according to the invention, which can be combined with all other embodiments, in step a) benzene is used in a stoichiometric excess, based on nitric acid, in the range from 2.0% to 40%, preferably 3.0% to 30%, particularly preferably 4.0% to 25%, of theory.

In a third embodiment of the process according to the invention, which can be combined with all other embodiments, the temperature in each of the n reactors of step a) is maintained in the range from 98° C. to 140° C.

In a fourth embodiment of the process according to the invention, which can be combined with all other embodiments, the process comprises the following:
(α) after the combining in step b)(i), introducing the mixed stream containing nitrobenzene, benzene and sulfuric acid into a gas separator in which a gaseous phase comprising benzene and gaseous secondary components is removed and a liquid phase comprising nitrobenzene and sulfuric acid and depleted of gaseous constituents remains and is fed to step b)(ii);
   or
(β) after step a) and before the combining in step b)(i), introducing the n process products containing nitrobenzene, benzene and sulfuric acid into n gas separators in which n gaseous phases comprising benzene and gaseous secondary components are removed and n liquid phases comprising nitrobenzene and sulfuric acid and depleted of gaseous constituents remain and are then fed to step b)(i);
   or
(γ) for carrying out the combining in step b)(i), introducing the n process products containing nitrobenzene, benzene and sulfuric acid from step a) into a common gas separator in which a gaseous phase comprising benzene and gaseous secondary components is removed and the mixed stream remains as liquid phase comprising nitrobenzene and sulfuric acid and depleted of gaseous constituents, which is fed to step b)(ii).

In a fifth embodiment of the process according to the invention, which is a particular configuration of the fourth embodiment, gravitational separators or centrifugal separators are used for removing the gaseous phase(s) comprising benzene and gaseous secondary components.

In a sixth embodiment of the process according to the invention, which is a particular configuration of the fifth embodiment, gravitational separators are used.

In a seventh embodiment of the process according to the invention, which is a particular configuration of the sixth embodiment, horizontally or vertically arranged gravitational separators are used, to which the process products containing nitrobenzene, benzene and sulfuric acid or the mixed stream containing nitrobenzene, benzene and sulfuric acid are respectively
fed from the side or from the bottom, wherein the gaseous phase is withdrawn from the gravitational separator as a top stream and the liquid phase is withdrawn from the gravitational separator as a bottom stream at the bottom or from the side, or
fed from the top, wherein the gaseous phase is withdrawn from the gravitational separator at the side and the liquid phase is withdrawn from the gravitational separator at the bottom.

In an eighth embodiment of the process according to the invention, which is a further particular configuration of the fifth embodiment, centrifugal separators are used.

In a ninth embodiment of the process according to the invention, which is a particular configuration of the eighth embodiment, the centrifugal separators used are vertically arranged, cylindrical, conical or cylindrical-conical cyclones through which the process product containing nitrobenzene, benzene and sulfuric acid or the mixed stream containing nitrobenzene, benzene and sulfuric acid is respectively guided with the generation of swirl, wherein the gaseous phase is discharged towards the top and the liquid phase is discharged towards the bottom.

In a tenth embodiment of the process according to the invention, which can be combined with all other embodiments, provided that they do not provide for a dividing of the mixed stream obtained in step b)(i), the entire mixed stream obtained in step b)(i) is fed to the phase separation apparatus of step b)(ii) at one location.

In an eleventh embodiment of the process according to the invention, which can be combined with all other embodiments, provided that they do not provide for the feeding of the unmodified entirety of the mixed stream obtained in step b)(i) into the phase separation apparatus at a single location, the mixed stream obtained in step b)(i) is divided into two or more (in particular into 2 to n, preferably into 2 to 3) substreams and these substreams are fed to the phase separation apparatus of step b)(ii) at various locations.

In a twelfth embodiment of the process according to the invention, which can be combined with all other embodiments, the n reactors in step a) are controllable independently of each other.

In a thirteenth embodiment of the process according to the invention, which can be combined with all other embodiments, the reactors used in step a) are tubular reactors, which are preferably arranged vertically and each have two or more (preferably in each case 2 to 15, particularly preferably 4 to 12, excluding the mixing device used for the initial mixing of benzene with nitric and sulfuric acid) dispersing elements, a flow through the tubular reactors particularly preferably being effected from bottom to top (i.e. the starting materials benzene-containing stream, sulfuric acid and nitric acid are fed to the vertically arranged tubular reactors in each case at the bottom and the process product containing nitrobenzene, benzene and sulfuric acid is withdrawn from the tubular reactors in each case at the top).

In a first embodiment of the production plant according to the invention, which can be combined with all other embodiments, the production plant has the following:

in a variant (α),
b) arranged downstream of the apparatus for combining the n process products containing nitrobenzene, benzene and sulfuric acid and upstream of the phase separation apparatus, a gas separator for separating the mixed stream from b)(i) into a gaseous phase comprising benzene and gaseous secondary components and a mixed stream comprising nitrobenzene, benzene and sulfuric acid and depleted of gaseous constituents,
   or, in a variant (β),
b) arranged downstream of the n reactors of a) and upstream of the apparatus for combining the n process products containing nitrobenzene, benzene and sulfuric acid, n gas separators operated in parallel for separating the process products of the n reactors of a) into n gaseous phases comprising benzene and gaseous secondary components and n process products comprising nitrobenzene, benzene and sulfuric acid and depleted of gaseous constituents,
   or, in a variant (γ),
b) arranged downstream of the n reactors of a), one such apparatus for combining the n process products containing nitrobenzene, benzene and sulfuric acid, which also functions as a gas separator for separating the process products of the n reactors of a) into a gaseous phase comprising benzene and gaseous secondary components and a mixed stream comprising nitrobenzene, benzene and sulfuric acid and depleted of gaseous constituents.

In a second embodiment of the production plant according to the invention, which can be combined with all other embodiments, the n reactors of a) are controllable independently of each other.

In a third embodiment of the production plant according to the invention, which can be combined with all other embodiments, provided that they do not provide for a division of the mixed stream from b)(i), the phase separation apparatus has a single inlet connection for introducing the entire mixed stream.

In a fourth embodiment of the production plant according to the invention, which can be combined with all other embodiments, provided that they do not provide for the feed of the unmodified entirety of the mixed stream from b)(i) into the phase separation apparatus at a single location, between the apparatus for combining the n process products containing nitrobenzene, benzene and sulfuric acid and the phase separation apparatus, there is arranged a distributor system for distributing the mixed stream to two or more (in particular 2 to n, preferably to 2 to 3) inlet connections fitted to the phase separation apparatus.

In a fifth embodiment of the production plant according to the invention, which can be combined with all other embodiments, the reactors of a) are tubular reactors, which are preferably arranged vertically and each have two or more (preferably in each case 2 to 15, particularly preferably 4 to 12, excluding the mixing device used for the initial mixing of benzene with nitric and sulfuric acid) dispersing elements, a flow through the tubular reactors particularly preferably being effected from bottom to top (i.e. the starting materials benzene-containing stream, sulfuric acid and nitric acid are fed to the vertically arranged tubular reactors in each case at the bottom and the process product containing nitrobenzene, benzene and sulfuric acid is withdrawn from the tubular reactors in each case at the top).

The embodiments briefly outlined above and further possible configurations of the invention are elucidated in more detail hereinafter. The abovementioned embodiments and further possible configurations may be combined with one another as desired, unless the opposite is apparent from the context.

Step a) of the process according to the invention, the nitration of a benzene-containing stream (henceforth also referred to as a.1) in n reactors with sulfuric acid (henceforth also referred to as a.2) and nitric acid (henceforth also referred to as a.3) using a, based on nitric acid (henceforth also referred to as a.3), stoichiometric excess of benzene, can in principle be conducted by all adiabatically operated nitration processes known from the prior art. According to the invention, two to five reactors, preferably two to three reactors, are operated in parallel.

It is preferable to first meter the nitric acid (a.3) and then the benzene-containing stream (a.1) into the sulfuric acid (a.2). The premixing of nitric acid (a.3) and sulfuric acid (a.2) produces the so-called mixed acid into which in this embodiment the benzene-containing stream (a.1) is then metered in. In this case, the mixed acid used contains, based on the total mass of the mixed acid, preferably at least 2.0% by mass of nitric acid and at least 66.0% by mass of sulfuric acid, particularly preferably 2.0% by mass to 4.0% by mass of nitric acid and 66.0% by mass to 75.0% by mass of sulfuric acid.

The stoichiometric excess of benzene based on nitric acid (a.3) is preferably set to a value in the range from 2.0% to 40%, particularly preferably in the range from 3.0% to 30%, very particularly preferably in the range from 4.0% to 25%, of theory. Theoretically, 1 mol of HNO₃ reacts with 1 mol of benzene. A benzene excess of x % in relation to HNO₃ therefore corresponds to a molar ratio n(benzene)/n(HNO₃) (n=molar amount) of

$$\frac{1 + \frac{x}{100}}{1} ,$$

i.e. for example

$$\frac{1 + \frac{2}{100}}{1} = 1.02$$

with a 2% benzene excess or for example

$$\frac{1 + \frac{40}{100}}{1} = 1.40$$

with a 40% benzene excess.

It is preferable to recover excess benzene and use it in part or in full as a constituent of the benzene-containing stream (a.1). The excess benzene is recovered in this case before or after, especially after, a single- or multi-stage washing of the crude nitrobenzene; for further details reference can be made to the discussion of step c) hereinbelow. The benzene-containing stream (a.1) is therefore preferably a mixture of benzene freshly fed to the reaction (referred to as fresh benzene) and recycled benzene (referred to as return benzene). In any case, the reaction conditions are in particular selected so that the proportion by mass of benzene in the benzene-containing stream (a.1), based on the total mass of the benzene-containing stream (a.1), is at least 90.0%, preferably at least 95.0%, particularly preferably at least 98.5%.

According to the invention, step a) is conducted under adiabatic conditions. In the case of adiabatic reaction regime, the reactor used in step a) is neither heated nor cooled; the reaction temperature results from the temperature of the reactants used and the mixing ratio between them. The n reactors are preferably well insulated in order to reduce heat losses to a minimum. If the nitration is conducted adiabatically, the reaction temperature of the mixture reacting in each of the n reactors thus increases from the “starting temperature” immediately after the first mixing of the reactants up to the “end temperature” after maximum conversion and is preferably maintained constantly at values in the range from 98° C. to 140° C. The starting temperature results from the temperatures of the feedstocks benzene, sulfuric acid and nitric acid, from the concentrations of the acids used, from the quantitative ratio between them and from the volumetric ratio of organic phase (benzene) to aqueous phase (sulfuric and nitric acid), what is known as the phase ratio. The phase ratio is also decisive for the end temperature: The smaller the phase ratio (thus the more sulfuric acid present), the lower the end temperature. In the case of the preferred use of a tubular reactor (see hereafter), the temperature rises as a result of increasing conversion along the longitudinal axis of the reactor. At the entry into the reactor the temperature is in the lower region of the mentioned temperature range of 98° C. to 140° C., at the exit from the reactor the temperature is in the upper region of the mentioned temperature range.

Preferably, step a) is executed in a process regime as described in DE 10 2008 048 713 A1, especially paragraph [0024].

Suitable reactors for step a) are in principle any reactors known in the prior art for adiabatic nitrations, such as stirred tanks (especially stirred tank cascades) and tubular reactors. Tubular reactors are preferred. Particular preference is given here to a tubular reactor in which two or more dispersing elements are distributed over the length of the tubular reactor, these ensuring intense mixing of benzene, nitric acid and sulfuric acid. Particular preference is given to using a vertically arranged tubular reactor in which two or more (preferably 2 to 15, particularly preferably 4 to 12, excluding the mixing device used for the initial mixing of benzene with nitric and sulfuric acid) dispersing elements are distributed over the length of the tubular reactor. The flow through such a tubular reactor is very particularly preferably from bottom to top. Such a reactor, and the form of usable dispersing elements, are described for example in EP 1 291 078 A2 (see there FIG. 1).

It is particularly preferable to configure step a) so that the n reactors are controllable independently of each other, that is to say can be operated independently of each other. This allows production output to be made possible through shutdown of individual reactors when there is a reduced demand for the product nitrobenzene.

In step b) of the process according to the invention, the n process products containing nitrobenzene, benzene and sulfuric acid (and also secondary components which may be present as gas phase or in dissolved form) (henceforth also referred to as a.4.1, a.4.2, . . . , a.4.n) of step a) are first combined in a step b)(i) into a mixed stream containing nitrobenzene, benzene and sulfuric acid (henceforth also referred to as b.1). This is effected in an apparatus for combining the process products (a.4.1, a.4.2, . . . a.4.n) containing nitrobenzene, benzene and sulfuric acid obtained in the n reactors. Such an apparatus is for example a vessel which is connected via lines to the exit openings for the crude process products of the nitration from the reactors (shown in FIG. 1a using the example with n=2). The n crude process products of the nitration are combined in this vessel. The combining can also be effected in a pipeline into which the n streams (a.4.1, a.4.2, . . . a.4.n) jointly flow and the mixed stream (b.1) of which is passed to step c) (shown in FIG. 1b using the example with n=2). It is also possible to support the desired mixing (=homogenization) of the n crude process products by using mixing-promoting static internals or a stirrer in the apparatus for combining the n crude process products.

In a preferred embodiment, a gas-liquid phase separation for depleting gaseous constituents takes place before the phase separation in step b)(ii). This gas-liquid phase separation is effected in a gas separator. Gas separators which can be used are in principle all separators known to those skilled in the art which enable a gas-liquid separation. Possible apparatuses for the separation of gaseous and liquid streams are general knowledge for those skilled in the art. Details concerning the various processes and equipment for separating gaseous and liquid streams can be found in the specialist literature, such as for example in Oilfield Processing, Crude Oil, Vol. 2, chapter 6, page 79 to 112, year 1995, by Manning, Francis S. and Thompson, Richard E. or in Gulf Equipment Guides, Gas-Liquid and Liquid-Liquid Separators, chapters 3.3 to 3.5 on pages 72 to 103, year 2009, by Maurice Stewart and Ken Arnold. The variants described in the cited literature are explained in part using the example of triphasic gas-liquid-liquid separations, but are also usable for gas-liquid phase separations as concerns the fundamental principles. This preferred embodiment features the separation of the steps (i) removal of (at least the majority of) the gas phase from the two liquid phases and (ii) separation of the two liquid phases from each other. Therefore, these steps are performed in this embodiment in two apparatuses, the gas separator and the phase separation apparatus. However, in terms of the apparatus configuration, the gas separator and the phase separation apparatus may by all means share common features.

The gas separator is preferably not temperature-controlled, as a result of which the temperatures in the gas separator result from the temperature of the inflowing reaction mixture. The gas separator is preferably operated at slightly elevated pressure with respect to ambient pressure (“positive pressure”), the pressure in the gas space of the gas separator being 50 mbar to 100 mbar, for example 80 mbar, above ambient pressure.

Preference is given to using gravitational separators or centrifugal separators as gas separator.

The gas-liquid phase separation can be implemented in various ways.

In a variant (α), the gas-liquid phase separation is effected after the combining of the n crude process products of the nitration of step b)(i) described above. Here, the mixed stream (b.1) containing nitrobenzene, benzene and sulfuric acid is introduced into a gas separator in which a gaseous phase comprising benzene and gaseous secondary components (henceforth also referred to as b.3) is removed and a liquid phase comprising nitrobenzene and sulfuric acid and depleted of gaseous constituents (henceforth also referred to as b.2) remains and is fed to step b)(ii) as mixed stream for separation into a sulfuric acid phase and a nitrobenzene phase.

In a variant (β), the gas-liquid phase separation is effected after step a) (and before the combining of then crude process products of the nitration of step b)(i)). Here, then process products (a.4.1, a.4.2, . . . a.4.n) containing nitrobenzene, benzene and sulfuric acid are passed into n gas separators in which n gaseous phases comprising benzene and gaseous secondary components (henceforth also referred to as b.3.1, b.3.2, b.3.n) are removed and n liquid phases comprising nitrobenzene and sulfuric acid and depleted of gaseous constituents (henceforth also referred to as b.2.1, b.2.2, . . . b.2.n) remain and are then fed to the combining of the n crude process products of the nitration of step b)(i).

In a variant (γ), the gas-liquid phase separation and the combining of the n crude process products of the nitration are effected in a common apparatus, i.e. the gas-liquid phase separation is a particular form of the combining of the n crude process products of the nitration of step b)(i). Here, the n process products (a.4.1, a.4.2, a.4.n) containing nitrobenzene, benzene and sulfuric acid from step a) are passed into a common gas separator in which a gaseous phase comprising benzene and gaseous secondary components is removed and the mixed stream remains as liquid phase comprising nitrobenzene and sulfuric acid and depleted of gaseous constituents, which is fed to step b)(ii). In this variant, spontaneous evaporation may occur under certain conditions, if the individual liquid fractions of the process products (a.4.1, a.4.2, a.4.n) mix and have markedly different compositions and/or temperatures. Such a spontaneous evaporation could interfere with the homogenization and the gas-liquid phase separation.

Preference is therefore given to the variants (α) and (β). The homogenization of the nitrated reaction solutions from the individual reaction lines prior to the liquid-liquid phase separation leads to a reduction or avoidance of turbulence on entry into the phase separation apparatus of step b)(ii). Undesired flows in the apparatus, such as crossflows and backflows and also swirl formation, which form due to different proportions of the three phases (aqueous, organic, gas) in the incoming reaction solutions, can be reduced or eliminated. The same also applies to the gas separator. Combining prior to the gas-liquid phase separation is the simplest in terms of apparatus, and accordingly particular preference is given to the variant (α).

Irrespective of the variant chosen, in one embodiment of the invention the gas separators used may be horizontally or vertically arranged gravitational separators to which the process products (a.4.1, a.4.2, . . . a.4.n) containing nitrobenzene, benzene and sulfuric acid or the mixed stream (b.1) containing nitrobenzene, benzene and sulfuric acid are respectively
fed from the side or from the bottom, wherein the gaseous phase comprising benzene and gaseous secondary components is withdrawn from the gravitational separator as a top stream and the liquid phase comprising nitrobenzene and sulfuric acid and depleted of gaseous constituents is withdrawn from the gravitational separator as a bottom stream at the bottom or from the side, or
fed from the top, wherein the gaseous phase comprising benzene and gaseous secondary components is withdrawn from the gravitational separator from the side and the liquid phase comprising nitrobenzene and sulfuric acid and depleted of gaseous constituents is withdrawn from the gravitational separator at the bottom.

The expression “horizontally or vertically arranged” relates to the longitudinal axis of the essentially cylindrical apparatus. FIG. 2 to FIG. 4 show vertically arranged gravitational separators which can be used in the gas-liquid separation step. For the sake of simplicity, the streams in the drawings are identified as for the variant (α) (feed stream=b.1; gaseous phase=b.3, remaining liquid phase=b.2):

In the gas separator according to FIG. 2, the process product (b.1) is fed from the side, the gas phase (b.3) is discharged at the top and the liquid phase (b.2) is discharged at the bottom.

In the gas separator according to FIG. 3, the process product (b.1) is fed at the bottom, the gas phase (b.3) is discharged at the top and the liquid phase (b.2) is discharged from the side.

In the gas separator according to FIG. 4, the process product (b.1) is fed at the top, the gas phase (b.3) is discharged from the side and the liquid phase (b.2) is discharged at the bottom.

Preference is given to a configuration according to FIG. 2.

However, it is also possible to use a centrifugal separator. Preference is given here to a vertically arranged, cylindrical, conical or cylindrical-conical cyclone through which the process product containing nitrobenzene, benzene and sulfuric acid is guided with the generation of swirl, wherein the gaseous phase comprising benzene and gaseous secondary components is discharged towards the top and the liquid phase comprising nitrobenzene and sulfuric acid and depleted of gaseous constituents is discharged towards the bottom. The term “vertically arranged” again relates to the longitudinal axis of the apparatus. The swirl can be generated either through a tangentially arranged entry connection or a deflecting plate (see FIG. 3.20 in Gulf Equipment Guides: Gas-liquid and liquid-liquid Separators, Stewart & Arnold, 2009, Gulf Professional Publishing).

In step b)(ii), the mixed stream obtained in step b)(i) is passed into a phase separation apparatus. In the simplest configuration of this step, this can be accomplished by feeding the mixed stream to the phase separation apparatus via a single inlet connection (i.e. the entire mixed stream obtained in step b)(i) is fed to the phase separation apparatus of step b)(ii) at one location, as illustrated in FIG. 5 and FIG. 6). In this case it is recommended to dimension the inlet connection sufficiently large to be able to feed the one single, relatively large (compared to the individual streams in the mode without combining) mixed stream to the phase separation apparatus without encountering high flow velocities and swirling in the phase separation apparatus.

If the intention is to retroactively introduce the procedure according to the invention into an already existing production plant having n parallel-connected reactors and accordingly also having n—arranged at various spatial locations of the phase separation apparatus—inlet connections for the n crude process products, it is preferable to continue to use the already present n inlet connections of the phase separation apparatus and the associated pipelines by connecting the apparatus to be used according to the invention for combining the n crude process products (a.4.1, a.4.2, . . . a.4.n) between the n reactor exits and the n entrances into the phase separation apparatus. In this case, the mixed stream (b.1) departing the apparatus for combining the n crude process products (a.4.1, a.4.2, . . . a.4.n) can either be divided again into n substreams, or the apparatus for combining the n process products is provided, in a departure from FIG. 1, with n exit connections (in which case it should be ensured that the n crude process products are sufficiently mixed (=homogenized) on reaching the exit connections, for example by dimensioning the apparatus sufficiently large in order to provide adequate residence time and/or by using mixing-promoting static internals or a stirrer). That is to say, if the combining according to the invention in the context of step b)(i) is conducted, then at the end of this step only a single, uniform process product is present having a given temperature and chemical composition (the mixed stream). By again dividing this uniform process product into substreams, the temperature and composition of the individual substreams are not altered further with respect to the single uniform process product, meaning that this procedure does not detract from the inventive concept. Therefore, with this procedure, too, the substreams fed to the phase separation apparatus are in homogenized form with respect to their velocities, temperatures and chemical compositions at the entrances to the phase separation apparatus.

The procedure using two or more inlet connections into the phase separation apparatus further has the advantage that the velocities at the individual inlet connections (for an identical diameter) and also in general the inlet and mixing processes are markedly reduced, and the phase separation can begin more rapidly. It can therefore also be expedient, when planning a new production plant, to divide the mixed stream containing nitrobenzene, benzene and sulfuric acid, obtained in step b)(i), into two or more, in particular 2 to n, preferably 2 to 3, substreams (in the same manner as described above), and to feed these to the phase separation apparatus at spatially separate locations.

Irrespective of whether the mixed stream is fed to the phase separation apparatus in its unmodified entirety at one location or is fed divided into two or more substreams at two or more different locations, the liquid-liquid phase separation in step b)(ii) can be effected according to processes known per se from the prior art in a phase separation apparatus known in principle to those skilled in the art. The aqueous sulfuric acid phase (henceforth also referred to as b.5) essentially contains (as a result of the formation of water of reaction and due to the introduction of water into the reaction from the nitric acid used) diluted sulfuric acid alongside inorganic impurities. The organic nitrobenzene phase (henceforth also referred to as b.4) essentially contains nitrobenzene alongside excess benzene and organic impurities. The phase separation apparatus is preferably provided with a gas outlet, via which any gaseous constituents present can be discharged (to the extent that these have not already been removed beforehand in the preferred gas-liquid separation). The gas outlet of the optionally present gas separator and the gas outlet of the phase separation apparatus of step b)(ii) preferably open out into a common offgas workup apparatus. The phase separation apparatus of step b)(ii) is preferably not temperature-controlled and is preferably operated at a slight positive pressure (preferably 50 mbar to 100 mbar, for example 80 mbar, above ambient pressure, measured in the gas space).

Irrespective of the precise mode and the precise configuration of the reactor in step a) and of the apparatus for combining the n process products, of the gas separator optionally present and of the phase separation apparatus of step b), it is preferable to concentrate the liquid aqueous, sulfuric acid-comprising phase obtained in step b) (henceforth also referred to as b.5) by evaporation of water to give a liquid aqueous phase (henceforth also referred to as d.1) comprising a higher concentration of sulfuric acid compared to phase (b.5), to recycle it into step a) and to use it in part or in full as constituent of the sulfuric acid (a.2) used there. In this case, the sulfuric acid (a.2) used in step a) therefore contains recycled sulfuric acid (d.1) and in certain embodiments can even consist thereof. This preferred process regime is referred to in the terminology of the present invention as step d) and is explained in yet more detail below.

In step c) of the process according to the invention, the liquid phase obtained in step b)(ii) (henceforth also referred to as b.4) (the crude nitrobenzene) is worked up to obtain nitrobenzene (henceforth also referred to as c.1). This workup can in principle be accomplished as known in the prior art. A preferred procedure is outlined below:

First, the organic phase (b.4) is washed in one or more stages (step c)(i)). In a first substep of this wash, the organic phase (b.4), which typically still contains traces of acid, is washed in one or more stages with an aqueous washing liquid and then separated from the acidic aqueous phase obtained by phase separation, in the case of two or more washing stages after each individual washing stage. In this operation, the acid residues contained in the crude nitrobenzene (b.4) are washed out, this process step is therefore also referred to as acidic wash. This step is sufficiently well known from the prior art and is therefore outlined only briefly here. Preferably, for performance of this acidic wash, aqueous streams obtained in operation are recycled.

The organic phase thus obtained is then, in a second substep in an alkaline wash, washed in one or more stages with an aqueous solution of a base, preferably selected from sodium hydroxide, sodium carbonate or sodium hydrogencarbonate, and then separated from the alkaline wash water by phase separation, in the case of two or more washing stages after each individual washing stage. Particular preference is given to using sodium hydroxide solution as aqueous base solution. This step is sufficiently well known from the prior art and is therefore outlined only briefly here. The pH of the sodium hydroxide solution used and its mass ratio to the organic phase are adjusted such that acidic impurities (for example nitrophenols formed as by-products and acid residues incompletely removed in the first substep) are neutralized in the alkaline wash. The subsequent workup of the alkaline wastewater can be effected by the methods of the prior art, for example according to the teaching of EP 1 593 654 A1 and EP 1 132 347 A2.

The organic phase thus obtained is lastly, in a third substep in a neutral wash, washed in one or more stages with water and then separated from the aqueous phase by phase separation, in the case of two or more washing stages after each individual washing stage. This can in principle be accomplished by any methods that are customary in the prior art. The washing water used here is preferably demineralized water, more preferably a mixture of demineralized water and steam condensate (i.e. a condensate of steam which has been obtained by heat exchange of water with any exothermic process steps), and most preferably steam condensate. Preference is given to a procedure in which an electrophoresis is used in the last neutral stage of the neutral wash (see WO 2012/013678 A2).

The nitrobenzene washed in this way is lastly freed of dissolved water, unconverted benzene and any organic impurities by further workup (step c)(ii)). This workup is preferably effected by distillation, wherein the vapors of water and benzene and any organic impurities are driven off overhead. The vapors are cooled and run into a separating vessel. Water separates out in the lower phase and is removed. In the upper phase are benzene and low boilers, which are fed back to the reaction as return benzene (c.2). If necessary, a portion of this upper phase can be discharged (that is to say, not recycled) in order to avoid excessive accumulation of low boilers. It is also possible to separate low boilers off from this upper phase and to feed a return benzene depleted of low boilers to the reaction. The distillation apparatus used is preferably a rectification column. The bottom product from the distillation, optionally after a further distillation in which nitrobenzene is obtained as distillate (i.e. as topstream or sidestream product), is sent to further applications (such as in particular hydrogenation to aniline) as (pure) nitrobenzene (c.1).

Alternatively to the procedure presented here, it is also conceivable to remove excess benzene prior to the wash.

As already mentioned, it is preferable in a step d) to concentrate the liquid aqueous, sulfuric acid-comprising phase (b.5) obtained in step b)(ii) by evaporation of water to give a liquid aqueous phase (henceforth also referred to as d.1) comprising a higher concentration of sulfuric acid compared to phase (b.5), to recycle it in part or in full into step a) and to use it as constituent of the sulfuric acid (a.2) used there. This concentration of the aqueous sulfuric acid phase (b.5) can in principle be effected as known from the prior art. Preference is given to an embodiment in which the sulfuric acid in the aqueous phase (b.5) is concentrated in a flash evaporator by evaporating water into a region of reduced pressure. In the adiabatic mode provided according to the invention it is possible, given correct choice of the reaction conditions, to achieve such significant heating in step a) of the sulfuric acid-containing aqueous phase (b.5) with the heat of reaction of the exothermic reaction that, in the flash evaporator, the concentration and temperature of the sulfuric acid-containing aqueous phase that it had prior to the reaction with benzene and nitric acid on entry into the reactor space can simultaneously be established again, that is to say (d.1) corresponds to (a.2) in terms of temperature and concentration. This is described in EP 2 354 117 A1, especially paragraph [0045].

As already mentioned, the present invention secondly provides a production plant for performing the process according to the invention for the continuous preparation of nitrobenzene. Preferred embodiments and configurations of the process according to the invention apply likewise correspondingly to the production plant according to the invention. For example, the production plant according to the invention preferably comprises tubular reactors as reactors.

The appended drawing FIG. 5 shows a possible embodiment of the production plant according to the invention using the example where n=2. The following references apply in the drawing:
1001, 1002: Reactors
2100: Apparatus for combining the process products obtained in the reactors
2200: Phase separation apparatus
3000: Device for sulfuric acid concentration (evaporator)
4000: Sulfuric acid tank
5000: Crude nitrobenzene tank
6000: Devices for single- or multi-stage washing of the crude nitrobenzene
7000: Device for removing unconverted benzene (in particular rectification column)

In one particular embodiment, the production plant according to the invention additionally comprises one or more gas separators. In this case, as already described above in connection with the process according to the invention, there are multiple options for the further configuration of the production plant:
(α) The production plant can have a gas separator arranged downstream of the apparatus for combining the process products containing nitrobenzene, benzene and sulfuric acid obtained in the n reactors (and upstream of the phase separation apparatus).
(β) However, it is also possible to connect, downstream of the n reactors operated in parallel, n gas separators operated in parallel, the liquid exits of which open into the apparatus for combining the process products containing nitrobenzene, benzene and sulfuric acid obtained in the n reactors.
(γ) Lastly, the apparatus for combining the process products containing nitrobenzene, benzene and sulfuric acid obtained in the n reactors can be configured such that it performs the functions of combining the n crude process products of the nitration and of depleting gaseous constituents jointly.

In the embodiment with additional gas-liquid separation, the production plant according to the invention therefore preferably comprises

in a variant (α),
b) arranged downstream of the apparatus for combining the n process products containing nitrobenzene, benzene and sulfuric acid and upstream of the phase separation apparatus, a gas separator for separating the mixed stream from b)(i) into a gaseous phase (b.3) comprising benzene and gaseous secondary components and a (liquid) mixed stream (b.2) comprising nitrobenzene, benzene and sulfuric acid and depleted of gaseous constituents, or, in a variant (β),
b) arranged downstream of the n reactors of a) and upstream of the apparatus for combining the n process products containing nitrobenzene, benzene and sulfuric acid, n gas separators operated in parallel for separating the process products of the n reactors of a) into n gaseous phases (b.3.1, b.3.2, . . . b.3.n) comprising benzene and gaseous secondary components and n (liquid) process products (b.2.1, b.2.2, . . . b.2.n) comprising nitrobenzene, benzene and sulfuric acid and depleted of gaseous constituents,
   or, in a variant (γ),
b) arranged downstream of the reactors of a), one such apparatus for combining the n process products containing nitrobenzene, benzene and sulfuric acid, which also functions as a gas separator (common to all n reactors) for separating the process products (a.4.1, a.4.2, a.4.n) of the n reactors of a) into a gaseous phase (b.3) comprising benzene and gaseous secondary components and a (liquid) mixed stream (b.2) comprising nitrobenzene, benzene and sulfuric acid and depleted of gaseous constituents.

Variant (a) is particularly preferred (see in this respect the corresponding statements further above in connection with the description of the process according to the invention) and is illustrated in FIG. 6 using the example of two reactors 1001 and 1002. Control valves and the like are not illustrated so as not to complicate the drawing. By combining the individual process products (a.4.1, a.4.2) in a vessel (2100) upstream of the gas separator (2110) and hence also upstream of the phase separation apparatus (2200), the construction of the phase separation apparatus is simplified (only one opening instead of at least two for metering in the liquid phase). In addition, the phase separation is facilitated since undesired flows in the apparatus such as crossflows and backflows and also swirl formation, which form due to different proportions of the three phases (aqueous, organic, gas) in the incoming reaction solutions, are reduced or eliminated. Moreover, varying throughputs and reaction conditions in the individual lines in the case of two or more entry openings lead to varying and highly differing velocities at the entrances and hence to unknown flow conditions in the phase separation apparatus. These influences can be better controlled by prior homogenization with joint metered addition at one location.

If the depletion of gaseous constituents according to one of the variants (α), (β) or (γ) is dispensed with, in a preferred embodiment gaseous constituents are to a certain extent removed in the phase separation of step b)(ii) by providing the phase separation apparatus with a gas outlet via which gaseous constituents are discharged. This is indicated in FIG. 5 by the arrow “b.3” at the upper end of the phase separation apparatus (2200). The gas outlet of the phase separation apparatus of step b)(ii) preferably opens into an offgas workup apparatus.

In all embodiments of the production plant according to the invention, it is particularly preferable to configure the production plant so that the n reactors are controllable independently of each other, that is to say can be operated independently of each other. This allows production output to be made possible through shutdown of individual reactors when there is a reduced demand for the product nitrobenzene. The devices required for this (in particular control valves and the corresponding controllers therefor) are sufficiently well known to those skilled in the art.

In the simplest configuration of introducing the mixed stream into the phase separation apparatus, the phase separation apparatus has a single inlet connection for the mixed stream.

If the mixed stream obtained in the apparatus for combining the process products (2100) obtained in the reactors, as described above as a possible embodiment in connection with the description of the process according to the invention, in a departure from the illustrations in FIG. 5 and FIG. 6, is intended to be divided into two or more (in particular 2 to n, preferably 2 to 3) substreams and fed to the phase separation apparatus (2200) at various locations, the production plant according to the invention has, downstream of the apparatus for combining the process products (2100) obtained in the reactors, a distributor system having a number of outlets corresponding to the number of substreams, and the phase separation apparatus (2200) has a plurality of inlet connections which are connected to the outlets of the distributor system and the number of which corresponds to the number of substreams. Such a distributor system can be realized simply by having the line for discharging the mixed stream from the apparatus for combining the process products (2100) obtained in the reactors open into two or more lines, the number of which corresponds to the number of substreams desired, or by having the apparatus for combining the process products (2100) obtained in the reactors possess a number of exit connections which corresponds to the number of substreams, the exit connections being connected to the inlet connections of the phase separation apparatus via lines.

The procedure according to the invention gives rise at least to the following advantages:
i) as a result of the combining of the individual reaction products of the n lines upstream of the phase separation apparatus and the associated homogenization, undesirable flows and turbulence in the phase separation apparatus can be minimized
ii) the n reactors can be operated independently of each other under differing process conditions (throughput, pressure, temperature), without this having negative effects on the separation performance of the phase separation apparatus.
iii) as a result of the degassing of the reaction solutions upstream of the phase separation apparatus which is performed in a preferred configuration of the invention, velocities and turbulence in the entry region of the phase separation apparatus are reduced, which can markedly increase the separating efficiency.
iv) the phase separation times in the phase separation apparatus are minimized, as a result of which the investment costs for this apparatus become lower, or a production increase in an existing plant becomes easier.
v) as a result of the improved phase separation, the entrainment of organics into the evaporator of the sulfuric acid concentration is reduced, which reduces energy consumption and avoids the problems otherwise caused by these organics.
vi) as a result of the improved phase separation, the entrainment of sulfuric acid in the crude nitrobenzene sent for workup is reduced. This brings about savings in feedstocks since the sulfuric acid losses in the workup turn out to be lower.
vii) the wastewater pollution is reduced as less sulfuric acid passes into the wastewater of step c).
viii) flow-stabilizing internals in the phase separation apparatus, which are prone to disruptive soiling and caking, can generally be dispensed with.

The present invention shall be illustrated below by means of examples.

### EXAMPLES

In the following two examples, the positive influence of homogenization of the incoming streams on the phase separation is to be made clear. To this end, Computational Fluid Dynamics simulations were performed of the triphasic flow behavior in the phase separation apparatus (apparatus 2200 in FIG. 5). In the examples discussed, three reactors are operated in parallel, the flows exiting therefrom flowing into the phase separation apparatus at three different locations. It was assumed for the simulation that the three reaction products (a.4.1, a.4.2, a.4.3) are obtained in different amounts since the three reactors are operated with differing production capacity. While the reaction product of the first reactor contains 300 t/h of aqueous phase (sulfuric acid phase), 17 t/h of organic phase (nitrobenzene phase) and 0.18 t/h of gas phase (predominantly benzene), from each of the other two reactors 225 t/h of aqueous phase (sulfuric acid phase), 13 t/h of organic phase (nitrobenzene phase) and 0.13 t/h of gas phase exit from the reaction. The phase separation apparatus is operated at an absolute pressure (in the gas phase) of approx. 1.1 bar, and the incoming streams have a temperature of 130° C.

FIG. 7 illustrates the employed 3D grid of the phase separation apparatus, with 800 000 computational cells. The following references apply in the drawing:
100: Inlet connections (3×; the third one is behind the observation plane and is not visible in the drawing)
200: Gas phase exit
300: Organic phase outflow
400: Aqueous phase outflow

For the sake of simplicity, the phase separation apparatus has been depicted as a cylinder, without considering curvatures of the lateral covers. Due to the axial symmetry, only half of the phase separation apparatus needs to be modeled. The process products of the three reactors (a.4) flow in via inlet connections on the left-hand side. The outflow of the organic phase (b.4) is situated in the middle on the right-hand side. The outflow of the aqueous phase (b.5) is situated at the lower end. The gas phase (b.3) can be taken off at the top. The triphasic flow was simulated using a Euler-Euler approach, the aqueous phase having been described as the continuous phase and the organic phase and the gas phase having been described as the disperse phase. The continuity and conservation of momentum equations were solved for all phases in the context of the simulation. The turbulence model used was a k-epsilon model. The equations were solved transiently, the time steps having been varied between 0.1 s and 0.001 s.

Since the exact droplet/bubble sizes on entry into the phase separation apparatus or else in the phase separation apparatus itself are not known, a constant droplet/bubble diameter of 1 mm was assumed for both phases. Since droplets and bubbles in reality follow a certain size distribution and breakup and coalescence processes take place in the apparatus, the actual particle sizes and the resulting phase proportions in the apparatus may vary. The objective of the CFD simulation is to qualitatively describe the influence of a gas phase on the flow conditions and ultimately the separating efficiency of the phase separation apparatus.

The examples respectively consider the case without homogenization (example 1) and with homogenization (apparatus 2100, examples 2 and 3).

### Example 1 (Comparative Example)

In comparative example 1, the individual lines with their respective amounts enter the phase separation apparatus at the three inflow connections.
300 t/h of aqueous phase, 17 t/h of organic phase and 0.18 t/h of gas phase from reactor 1;

225 t/h of aqueous phase, 13 t/h of organic phase and 0.13 t/h of gas phase from each of reactors 2 and 3.

The process product of the reactor with the greatest load flows in from the side at the central inlet. The process products of the two reactors with low load flow in from the side at the connections.

FIG. 8 illustrates the volume fractions of the three phases in gray scale (top image: volume fractions of aqueous phase, middle image: volume fractions of organic phase, bottom image: volume fractions of gas phase). In the images, the individual phases are also identified with
10: aqueous phase,
20: organic phase,
30: gas phase and
40: disperse phase in which complete mixing has not yet taken place.

It can be seen in the upper image that directly after entry a continuous aqueous phase forms which settles to the bottom. However, the aqueous phase is situated very far above the entry connection[KS1][CD2] and is initially entrained upwards by the rising gas stream. A continuous organic phase only forms at the end at the outflow (300) of the organic phase, the aqueous-organic phase boundary being located at the lower end of the outflow (middle image, coherent organic phase on the right-hand side upstream of the outflow of the organic phase 300), meaning that there is massive entrainment of aqueous phase. In the middle of the phase separation apparatus there is a large region in which all three phases are present (“disperse” phase) and swirling is visible. It is noticeable that the organic phase is present here only in very finely dispersed form and hardly reaches volume fractions of greater than 5%. In the lower image, in which the volume fractions of the gas phase are illustrated, it can be seen that the rising gas phase entrains the aqueous and organic phase upwards. As a result, the separation does not take place until late in the apparatus and the high volume fraction of aqueous phase and the barely visible proportions of organic phase can be explained by this. All in all, massive entrainment of extraneous phase at the individual outlets has to be assumed for such an operation, especially if in reality proportions of smaller droplet and gas bubble sizes than the 1 mm diameters simulated here are present, these requiring more time for separation.

In real production, the phase boundary can be observed through a sightglass fitted in the phase separation apparatus. Under the conditions described above, in real operation very marked fluctuations in the liquid-liquid phase boundary (±200 mm) were consistently observed on the right-hand side below the exit for the organic phase. In addition, rising gas bubbles are observed through the sightglass. The turbulence observed in the apparatus is all the more greater the more the loads of the individual reactors differ. In this mode, an aqueous phase was identified in the crude nitrobenzene tank (5000). The simulation is thus confirmed by the observations on the real apparatus.

### Example 2 (According to the Invention)

In example 2 according to the invention, the operation of the phase separation apparatus from example 1 was simulated taking into account upstream homogenization, that is to say it was assumed that the process products flowing into the phase separation apparatus via the three entry connections are identical in terms of temperature, composition and mass flow. In example 2, the process products of the individual reactors therefore enter the phase separation apparatus in equal proportions at the three inflow connections, specifically:
3×250 t/h of aqueous phase, 14 t/h of organic phase and 0.15 t/h of gas phase.

The results are illustrated in FIG. 9 (arrangement of the images and references as in FIG. 8). While a continuous organic phase still only forms towards the end of the phase separation apparatus (middle image, coherent organic phase on the right-hand side upstream of the outflow of the organic phase), the region has become much larger, and the aqueous-organic phase boundary is no longer located in the region of the outflow connection. A large region in which all three phases are present (“disperse” phase) remains in the middle of the phase separation apparatus. In this region, the proportion of organic phase has increased markedly, and so phase separation is already occurring here. This can also be seen in the middle and lower image, where larger volume fractions can also be seen in this region for the organic and gas phase. Overall, the organic phase and the gas phase are no longer dispersed so finely in the apparatus and the aqueous phase no longer passes upwards to as great an extent into the apparatus, and instead separates out downwards more rapidly. In the lower image, in which the volume fractions of the gas phase are illustrated, it can be seen that the gas phase rises upwards, yet a portion is still entrained far into the apparatus. Due to the low density of the gas phase (approx. 3 kg/m³) there is still a high volume fraction in the region of the entrance and in the middle part of the decanter, despite the low proportion by mass of the gas phase (0.15 t/h out of 250 t/h).

The high proportion of gas here also leads in the region of the entrance to higher velocities in the liquid phases (up to 2 m/s) and to swirl in the region of the liquid-liquid phase separation. For such an operation, entrainment of extraneous phase at the individual outlets still cannot be excluded, especially if in reality proportions of smaller droplet and gas bubble sizes than the 1 mm diameters simulated here are present, these requiring more time for separation.

All in all, the phase separation in the apparatus is markedly improved by the homogenization. Example 3 shows that this result can be improved further if gas separation is additionally performed.

### Example 3 (According to the Invention)

In example 3 according to the invention, the operation of the phase separation apparatus from example 2 was simulated taking into account upstream degassing. For this purpose, the proportion of the gas phase in each of the three process products (a.4.1, a.4.2, a.4.3) was reduced to 0.012 t/h (the simulation therefore assumes that >90% of the gas phase is removed, which is achievable without problems using conventional degassing apparatuses), which in real operation corresponds to variant (α) or (β). In the simulation in each case 250 t/h of aqueous phase and 14 t/h of organic phase continue to flow from each reactor into the phase separation apparatus. The volume fractions of the three phases are illustrated in FIG. 10 (arrangement of the images and references as in FIGS. 8 and 9). In contrast to example 2, it is apparent that a stable continuous aqueous and organic phase forms directly after entry into the phase separation apparatus. Due to the low proportion of gas phase, this no longer interferes with the separation process. The velocities in the region of the entrance are likewise markedly reduced (<1 m/s). Even for small droplet diameters, the flow is stabilized such that a rise and a phase separation are possible. For such an operation, entrainment of extraneous phase at the individual outlets can very substantially be excluded.

The positive effect was also demonstrated in real operation, where after installation of the gas separator the phase boundary in the phase separation apparatus could be stabilized and in addition no rising gas bubbles could be seen in the vicinity of the exit.

1. A process for continuously preparing of nitrobenzene, comprising:
a) nitrating benzene under adiabatic conditions with sulfuric acid and nitric acid using a stoichiometric excess of benzene, based on nitric acid, wherein the nitrating occurs in n parallel-connected reactors, where n is a natural number in the range from 2 to 5, so that n process products containing nitrobenzene, benzene and sulfuric acid are obtained;
b) combining the n process products containing nitrobenzene, benzene and sulfuric acid into one mixed stream containing nitrobenzene, benzene and sulfuric acid, optionally comprising a depletion of gaseous constituents (α) after, (β) before or (γ) during the combining operation,
(ii) introducing the mixed stream, which may be been depleted of gaseous constituents, into a phase separation apparatus in which the mixed stream is separated into a liquid aqueous sulfuric acid phase and a liquid organic nitrobenzene phase;
c) working up the nitrobenzene phase from step b) to obtain nitrobenzene;
and optionally
d) evaporating water from the sulfuric acid phase obtained in step b) to obtain a concentrated sulfuric acid phase, and using the concentrated sulfuric acid phase as a constituent of the sulfuric acid used in step a).

2. The process as claimed in claim 1, in which the workup of the nitrobenzene phase in step c) comprises:
(i) washing the nitrobenzene phase and removing unconverted benzene, and
(ii) using removed benzene as a constituent of the benzene used in step a).

3. The process as claimed in claim 1, in which in step a) benzene is used in a stoichiometric excess, based on nitric acid, in the range from 2.0% to 40% of theory.

4. The process as claimed in claim 1, in which the temperature in each of the n reactors of step a) is maintained in the range from 98° C. to 140° C.

5. The process as claimed in claim 1, comprising:
(α) after the combining in step b)(i), introducing the mixed stream containing nitrobenzene, benzene and sulfuric acid into a gas separator in which a gaseous phase comprising benzene and gaseous secondary components is removed and a liquid phase comprising nitrobenzene and sulfuric acid and depleted of gaseous constituents remains and is fed to step b)(ii);
or
(β) after step a) and before the combining in step b)(i), introducing the n process products containing nitrobenzene, benzene and sulfuric acid into n gas separators in which n gaseous phases comprising benzene and gaseous secondary components are removed and n liquid phases comprising nitrobenzene and sulfuric acid and depleted of gaseous constituents remain and are then fed to step b)(i);
or
(γ) for carrying out the combining in step b)(i), introducing the n process products containing nitrobenzene, benzene and sulfuric acid from step a) into a common gas separator in which a gaseous phase comprising benzene and gaseous secondary components is removed and the mixed stream remains as liquid phase comprising nitrobenzene and sulfuric acid and depleted of gaseous constituents, which is fed to step b)(ii).

6. The process as claimed in claim 1, in which the entire mixed stream obtained in step b)(i) is fed to the phase separation apparatus of step b)(ii) at one location.

7. The process as claimed in claim 1, in which the mixed stream obtained in step b)(i) is divided into two or more substreams that are fed to the phase separation apparatus of step b)(ii) at various locations.

8. The process as claimed in claim 1, in which the n reactors in step a) are controllable independently of each other.

9. The process as claimed in claim 1, in which the n reactors in step a) are tubular reactors.

10. A production plant configured to perform a process for continuously preparing nitrobenzene as claimed in claim 1, comprising:
a) n parallel-connected reactors configured to adiabatically nitrate benzene with sulfuric acid and nitric acid using a stoichiometric excess of benzene, based on nitric acid, where n is a natural number in the range from 2 to 5, wherein the n parallel-connected reactors are configured to obtain n process products containing nitrobenzene, benzene and sulfuric acid;
b) (i) arranged downstream of the reactors of a), an apparatus configured to combinefor combining the n process products containing nitrobenzene, benzene and sulfuric acid into one mixed stream containing nitrobenzene, benzene and sulfuric acid,
(ii) arranged downstream of the apparatus configured to combine the n process products containing nitrobenzene, benzene and sulfuric acid, a phase separation apparatus configured to separate the mixed stream obtained into a liquid aqueous sulfuric acid phase and a liquid organic nitrobenzene phase;
c) an apparatus configured to work up the liquid organic nitrobenzene phase from b)(ii) to give nitrobenzene; and
d) optionally, devices configured to concentrate the sulfuric acid phase from b)(ii) by evaporating water and devices to recycle concentrated sulfuric acid phase thus obtained into the n parallel-connected reactors.

11. The production plant as claimed in claim 10, having in a variant (α),
b) arranged downstream of the apparatus configured to combine the n process products containing nitrobenzene, benzene and sulfuric acid and upstream of the phase separation apparatus, a gas separator configured to separate the mixed stream from b)(i) into a gaseous phase comprising benzene and gaseous secondary components and a mixed stream comprising nitrobenzene, benzene and sulfuric acid and depleted of gaseous constituents,
or, in a variant (β),
b) arranged downstream of the n parallel-connected reactors and upstream of the apparatus configured to combine the n process products containing nitrobenzene, benzene and sulfuric acid, n gas separators configured to be operated in parallel and configured to separate the process products of the n parallel-connected reactors into n gaseous phases comprising benzene and gaseous secondary components and n process products comprising nitrobenzene, benzene and sulfuric acid and depleted of gaseous constituents,
or, in a variant (γ),
b) arranged downstream of the n parallel-connected reactors, one apparatus configured to combine for combining the n process products containing nitrobenzene, benzene and sulfuric acid and configured to separate the process products of the n parallel-connected reactors into a gaseous phase comprising benzene and gaseous secondary components and a mixed stream comprising nitrobenzene, benzene and sulfuric acid and depleted of gaseous constituents.

12. The production plant as claimed in claim 10, in which the n parallel-connected reactors are configured to be controllable independently of each other.

13. The production plant as claimed in claim 10, in which the phase separation apparatus has a single inlet connection configured to introduce the entire mixed stream into the phase separation apparatus.

14. The production plant as claimed in claim 10, in which, between the apparatus configured to combine the n process products containing nitrobenzene, benzene and sulfuric acid and the phase separation apparatus, there is arranged a distributor system configured to distribute the mixed stream to two or more inlet connections fitted to the phase separation apparatus.

15. The production plant as claimed in claim 10, in which the n parallel-connected reactors are tubular reactors.
